(19) United States
(12) Patent Application Publication
(10) Pub. No.: US 2026/0224224 A1
(43) Pub. Date: Aug. 6, 2026

(54) EMBOLIC COIL ANNEALED TO HAVE VARIABLE STIFFNESS

(71) Applicant: BOSTON SCIENTIFIC SCIMED, INC., MAPLE GROVE, MN (US)

(72) Inventors: Robert M. Wold, Maple Lake, MN (US); Mark Steven Smith, Coon Rapids, MN (US); Grigory Severyukhin, Zimmerman, MN (US)

(73) Assignee: BOSTON SCIENTIFIC SCIMED, INC., MAPLE GROVE, MN (US)

(21) Appl. No.: 19/463,502

(22) Filed: Jan. 29, 2026

Related U.S. Application Data
(60) Provisional application No. 63/751,559, filed on Jan. 30, 2025.

(51) Int. Cl. A61B 17/12; A61B 17/00

(52) U.S. Cl. CPC A61B 17/1215; A61B 2017/00526

(57) ABSTRACT

An embolic coil includes a single wire formed into a primary coil and then forming the primary coil into a secondary coil having a distal section and a proximal section, where the distal section of the secondary coil has a first stiffness and the proximal section has a second stiffness less than the first stiffness. The wire has a constant diameter along an entirety of its length and the primary coil has a constant diameter along an entirety of its length. A difference in stiffness between the distal and proximal sections is created by selective infrared heat treatment of the wire.

### CROSS-REFERENCE TO RELATED APPLICATIONS

This application claims the benefit of priority of U.S. Provisional Application No. 63/751,559 filed Jan. 30, 2025, the entire disclosure of which is hereby incorporated by reference.

### TECHNICAL FIELD

The disclosure pertains to medical devices and more particularly to embolic coils, and methods for making and using such medical devices.

### BACKGROUND

A wide variety of medical devices have been developed for medical use including, for example, medical devices utilized to create therapeutic vascular occlusions (embolizations) to prevent or treat pathological conditions in situ. These medical devices may be used in a variety of vessels, and are manufactured and used according to any one of a variety of different methods. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example embolic coil includes a single wire formed into a primary coil having a primary shape and a constant diameter along an entirety of its length, the single wire having a constant diameter along an entirety of its length, the primary coil formed into a secondary coil having a secondary shape different from the primary shape with a distal section and a proximal section, the distal section of the secondary coil having a first stiffness and the proximal section having a second stiffness less than the first stiffness, and wherein a difference in stiffness between the distal and proximal sections is created by selective infrared heat treatment.

Alternatively or additionally to the embodiment above, the single wire comprises a platinum-tungsten alloy.

Alternatively or additionally to any of the embodiments above, the first stiffness is approximately double the second stiffness.

Alternatively or additionally to any of the embodiments above, the secondary coil is treated with infrared radiation in the secondary shape to create the difference in stiffness between the distal and proximal sections.

Alternatively or additionally to any of the embodiments above, the embolic coil further includes a plurality of polymeric fibers attached to the secondary coil.

An example method of manufacturing an embolic coil includes forming a single wire into a primary coil, forming the primary coil into a secondary coil having a distal section and a proximal section, and selectively applying infrared radiation to the secondary coil to create a first stiffness in the distal section and a second stiffness in the proximal section, wherein the first stiffness is greater than the second stiffness.

Alternatively or additionally to the embodiment above, selectively applying infrared radiation comprises exposing the distal section to a first amount of infrared radiation, and exposing the proximal section to a second amount of infrared radiation greater than the first amount of infrared radiation.

Alternatively or additionally to any of the embodiments above, selectively applying infrared radiation comprises exposing the proximal section to infrared radiation for a first time period that is approximately twice as long as a second time period in which the distal section is exposed to infrared radiation.

Alternatively or additionally to any of the embodiments above, selectively applying infrared radiation includes positioning a reflective shield over the distal section of the secondary coil for at least a first time period while exposing the secondary coil to infrared radiation such that the reflective shield prevents absorption of the infrared radiation by the distal section of the secondary coil during infrared radiation exposure.

Alternatively or additionally to any of the embodiments above, the single wire is made of a platinum-tungsten alloy.

Alternatively or additionally to any of the embodiments above, the primary coil has a constant diameter along an entirety of its length.

Alternatively or additionally to any of the embodiments above, the single wire has a diameter of 0.076 mm (0.003 inches) or 0.102 mm (0.004 inches).

Alternatively or additionally to any of the embodiments above, the proximal section exhibits a first radial force of approximately 0.039 N (4 gf) and the distal section exhibits a second radial force of approximately 0.074 N (7.5 gf).

Alternatively or additionally to any of the embodiments above, the single wire has a constant diameter along an entirety of its length.

Alternatively or additionally to any of the embodiments above, the first stiffness is approximately double the second stiffness.

Alternatively or additionally to any of the embodiments above, selectively applying infrared radiation occurs when the secondary coil is disposed on a mandrel.

Alternatively or additionally to any of the embodiments above, the mandrel has a hollow core.

Alternatively or additionally to any of the embodiments above, further including attaching a plurality of polymeric fibers to the secondary coil after selectively applying infrared radiation to the secondary coil.

An example method of manufacturing an embolic coil includes forming a single wire into a primary coil, the single wire having a constant diameter along an entirety of its length, the primary coil having a constant outer diameter along an entirety of its length, forming the primary coil into a secondary coil having a distal section and a proximal section, and selectively applying infrared radiation to the secondary coil to create a first stiffness in the distal section and a second stiffness in the proximal section, wherein the first stiffness is greater than the second stiffness, wherein selectively applying infrared radiation includes exposing the proximal section of the secondary coil to infrared radiation for a longer time period than the distal section.

Alternatively or additionally to the embodiment above, exposing the proximal section of the secondary coil to infrared radiation for the longer time period is achieved by positioning a shield member over the distal section of the secondary coil during at least a portion of the longer time period.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1A is a side view of an example primary embolic coil on a mandrel;

FIG. 1B is an end view of the primary embolic coil and mandrel of FIG. 1A;

FIG. 2 shows an example embolic coil in a secondary shape after infrared treatment;

FIG. 3 illustrates a method of selectively applying infrared radiation to an embolic coil on a mandrel;

FIG. 4 is a graph illustrating radial force testing of various embolic coils; and

FIG. 5 is a cross-sectional view of another example embolic coil with a plurality of fibers attached.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Embolic coils are medical devices used in endovascular procedures to treat various vascular conditions. Embolic coils can be used for purposes including, for example, to close blood vessels and/or fill aneurysmal sacs. These coils may include wires formed into a primary shape and made from biocompatible materials such as platinum, platinum-iridium, or platinum-stainless steel alloys. The coil primary shape may be enhanced with secondary shaping to achieve specific therapeutic outcomes. During deployment, embolic coils may be delivered in their primary shape through a delivery catheter to a target vessel, where they move into a secondary shape and conform to the vessel architecture. The characteristics of these coils, including their outer diameter, flexibility, and structural properties, may play a role in their performance during navigation through tortuous vasculature and their final deployment configuration.

Embolic coils are often releasably attached to a delivery system, generally a wire, for delivery of the coil to the desired treatment site. Upon completion of satisfactory positioning, which may include partial or complete withdrawal back into the delivery catheter, the embolic coil may be detached from the delivery system and remain in the body after the delivery system is withdrawn. Another type of embolic coil is a pushable coil that is not attached to the delivery system and is simply pushed by a wire-like device through the delivery catheter for one-way deployment.

Embolic coils may be designed and built with specific performance requirements. In previous instances, an embolic coil may have either high anchoring forces for anchoring the coil in the vessel, or low radial force for occluding a vessel. An anchoring coil may be used for establishing the point of embolization that, once deployed, will create a back stop for other coils to be introduced and packed up against the anchoring coil. Soft coils and/or packing coils may be placed after the anchoring coil and are deployed to pack into the first coil to help reduce and cut off blood flow in that vessel. It often takes multiple coils at a treatment site to reduce the blood flow enough for thrombus formation and occlusion. Liquid and/or conformable embolics may also be used to achieve minimal to no blood flow through the vessel when deployed. Liquid and/or conformable embolics may be used with an anchoring coil and packing coils.

Both anchoring and soft/packing coils have conventionally been heat treated in the same manor. The primary wound coil is wrapped onto a secondary mandrel and loaded into an annealing oven. The mandrel and the wrapped coil remain in the oven until the temperature of the mandrel and coil equalize with the oven temperature. This makes the material properties of the coil the same throughout the entire length of the coil.

A variable stiffness coil that has a “stiff” distal section with a higher radial force for anchoring the coil to the vessel wall, with the remainder of the coil being “softer” with a lower radial force compared to the distal section has the advantage of allowing a single coil to establish the distal anchoring point in the vessel and a proximal section of the coil that will allow immediate packing of the vessel at or adjacent the anchor point. The level of stiffness may be based on radial force measurements. The ability to increase the distal coil radial force allows for more reliable embolization coil anchoring specifically in the proximal high flow arteries such as the Proximal Splenic Artery. The ability for the soft proximal segment to pack can accelerate a thrombus formation, reduce procedural time, minimize the need for additional packing coils, and enable the use of liquid embolics.

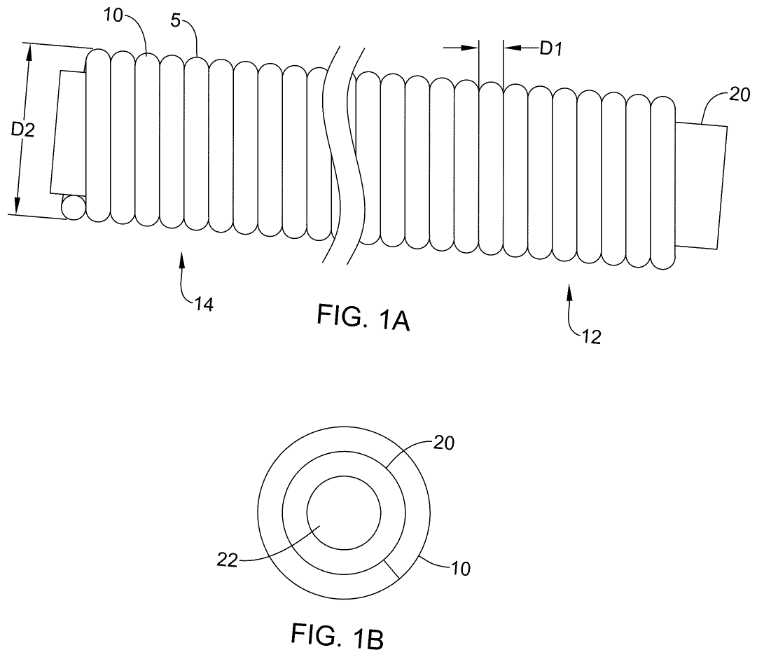

The inventors have developed a differential heat treatment that provides a variable stiffness along the length of a single coil. FIG. 1A is a side view of an example primary coil 5 on a mandrel 20 and FIG. 1B is an end view of the primary coil and mandrel of FIG. 1A. A single primary coil 5 may be formed from a single continuous wire 10 having a constant outer diameter D1 along its entire length that is wrapped around a first mandrel 20. The outer diameter D1 of the wire 10 may be between 0.002 inches and 0.005 inches (0.0508 mm to 0.127 mm), for example 0.003 inches (0.0762 mm) or 0.004 inches (0.1016 mm). The outer diameter D2 of the primary coil 5 may be between 0.015 inches to 0.030 inches (0.381 mm to 0.762 mm), and may be constant over the entire length of the primary coil 5. The overall length of the primary coil 5 may be between 0.5 cm to 100 cm (0.2 to 39.37 inches), for example 20 cm (7.87 inches).

The single wire 10 may be wound on a first mandrel 20 having a cylindrical shape with a constant diameter and a hollow core 22, as shown in FIG. 1B. In other embodiments, the first mandrel 20 may be a solid cylinder. In other embodiments, the first mandrel 20 may have a non-round cross-sectional shape, such as oval, stadium shape, egg shape, etc., and be solid or have a hollow core. The first mandrel 20 will generally have a constant diameter over its entire length, however in some embodiments, the mandrel may have a tapered diameter along its length. The single wire 10 may be wrapped around the first mandrel 20 with a constant pitch along the entire length of the primary coil 5. The single wire 10 may have a constant material composition along its entire length. In some embodiments, the single continuous wire 10 may be made of platinum and/or a platinum-containing alloy, including platinum-iridium, a platinum-tungsten alloy, platinum-stainless steel alloys, etc. In one embodiment, the single continuous wire 10 may be a platinum-tungsten alloy having a ratio of approximately 92:8 platinum to tungsten. In other embodiments, the primary coil 5 may be formed from multiple wires wrapped side by side or wrapped one over the other, where each wire has a constant diameter along its entire length and is formed from a constant material composition along its entire length.

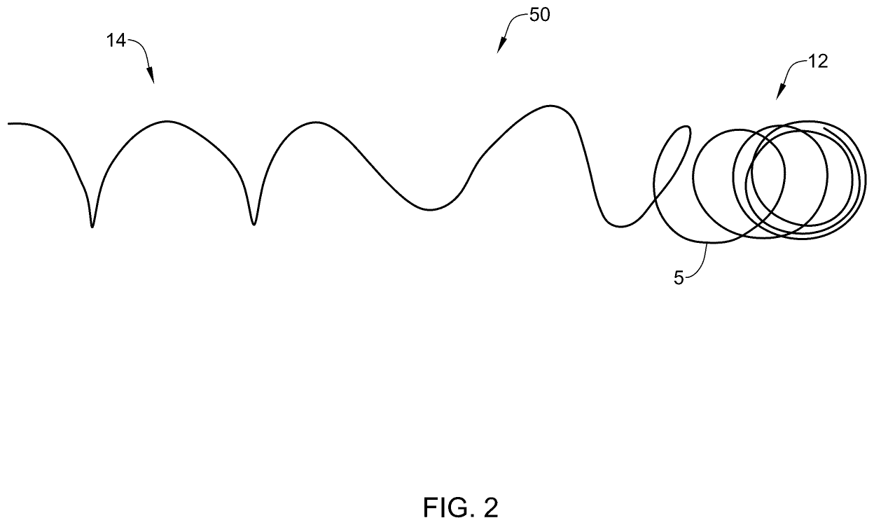

The single coil may be selectively annealed using infrared (IR) radiation. The variable stiffness in the single primary coil may be created using only IR annealing in selected regions. The single continuous wire 10 is wrapped around the first mandrel 20 to achieve a primary coil 5 with a primary shape, which may then be formed into a secondary coil 50 with a secondary shape different from the primary shape, which may then be selectively annealed using IR radiation to achieve regions of differing stiffness. FIG. 2 shows an example embolic coil 50 in a secondary shape. The secondary coil 50 may have a distal section 12 with a first stiffness and a proximal section 14 with a second stiffness less than the first stiffness. The first stiffness may be approximately double the second stiffness. After the primary coil 5 is formed into a secondary coil 50 and treated with IR radiation to achieve the variable stiffness, it maintains the secondary shape. In other embodiments, the embolic coil may be treated with IR radiation after the primary coil 5 has been formed but before winding the primary coil 5 into the secondary coil 50. The secondary coil 50 may be achieved by winding the primary coil 5 onto a larger cylindrical mandrel. In other embodiments, the secondary shape may be achieved by winding the primary coil 5 in planes perpendicular to the original diameter of winding on the first mandrel 20, achieving a cube shape. As shown in FIG. 2, after the primary coil 5 has been wound on a second mandrel larger than the first mandrel 20 and treated with IR radiation, the distal section 12 maintains its secondary shape and radial force while the proximal section 14 is more relaxed and compliant with the ability to pack easily within the vessel.

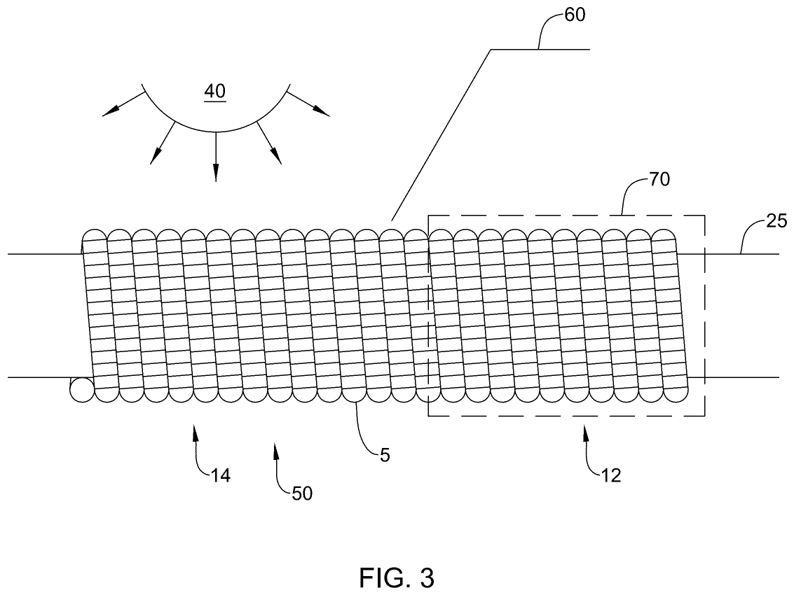

FIG. 3 illustrates a method of selectively applying infrared radiation to a secondary embolic coil 50 wound on a second mandrel 25 that is larger than the first mandrel 20. An IR radiation source (i.e., lamp) 40 may be used to anneal selected sections of the secondary coil 50 wrapped around the second mandrel 25. The IR source 40 may provide radiation over the entire IR spectrum, or just a portion thereof. The second mandrel 25 may have a hollow core (as shown for the hollow core first mandrel in FIG. 1i), causing the second mandrel 25 to absorb less energy than a solid mandrel, thus accelerating heat transfer to the secondary coil 50 when the secondary coil 50 and second mandrel 25 are subjected to heat treatment from the IR source 40. In other embodiments, the second mandrel 25 may be a solid cylinder. The second mandrel 25 may have a constant diameter, as illustrated in FIG. 3, or it may have a variable diameter.

Selected sections of the secondary coil 50 may be subjected to IR radiation for differing lengths of time and/or to achieve differing temperatures to achieve the desired level of stiffness. In some embodiments, the proximal section 14 of the secondary coil 50 may be exposed to IR radiation for a longer dwell time, such as double the time that the distal section 12 is exposed, so the wire in the proximal section 14 absorbs more IR energy, creating a softer section with a lower radial force in the proximal section 14 compared to the distal section 12. In some embodiments, shielding may be used to prevent the IR radiation from reaching other sections of the secondary coil 50. FIG. 3 illustrates two examples of shielding. For example, the distal section 12 of the secondary coil 50 may be covered by a polished reflective shield 60 while the proximal section 14 is exposed to the IR radiation source 40. In other embodiments, a ceramic sleeve 70 may be positioned over the distal section 12 while the proximal section 14 is uncovered and exposed to the IR radiation source 40. Any material that does not allow IR radiation absorption may be used as a shield 60 or sleeve 70.

A method of manufacturing the embolic coil includes forming a single wire 10 into a primary coil 5 having a distal section 12 and a proximal section 14. The single wire 10 may have a constant diameter along the entirety of its length. The primary coil 5 may be formed by winding the wire 10 around a first mandrel 20. The first mandrel 20 may have a continuous diameter along its length such that the primary coil 5 has a constant inner and outer diameter along its entire length. The primary coil 5 may then be formed into a secondary shape 50, such as by winding around a second mandrel 25 or folding the primary coil 5 into a secondary shape 50. In some embodiments, the second mandrel 25 may have a hollow core thereby absorbing less IR radiation energy than a solid mandrel. This may accelerate heat transfer to the secondary coil 50. In other embodiments, the second mandrel 25 may be a solid cylinder. IR radiation may then be applied selectively to the secondary coil 50 to create a first stiffness in the distal section 12 and a second stiffness in the proximal section 14, with the first stiffness being greater than the second stiffness. In some embodiments, the method may include exposing the distal section 12 to a first amount of IR radiation and exposing the proximal section 14 to a second amount of IR radiation greater than the first amount. The differing amounts of IR radiation may be achieved by exposing the proximal section 14 to IR radiation for approximately twice the duration as the distal section 12. For example, the proximal section 14 may be exposed to IR for two minutes and the distal section for one minute. The exposure times may alternatively be three minutes for the proximal section 14 and 1.5 minutes for the distal section 12.

The selective application of IR radiation may be achieved by positioning a reflective shield 60 over the distal section 12 of the secondary coil 50 for at least a first time period while exposing the entire secondary coil 50 to IR radiation such that the reflective shield prevents absorption of the IR radiation by the distal section 12 of the secondary coil 50 during IR radiation exposure. The shield may be a polished metal member 60 or a ceramic sleeve 70. After the first time period, the reflective shield or ceramic sleeve may be removed and the entire secondary coil 50 may be exposed to IR radiation for a second time period, resulting in the proximal section 14 being exposed to IR radiation for a third time period that is the combination of the first and second time periods. The first time period may be the same as the second time period. In other embodiments, the first time period may be half, 1.5 times or double the second time period. In addition to creating the differing stiffnesses in the distal section 12 and the proximal section 14, the IR radiation treatment heat sets the shape of the secondary coil 50. In this manner, when the embolic coil 50 is loaded into a delivery catheter in the primary shape, the coil expands as it exits the delivery catheter and assumes the secondary shape 50 after delivery. In some embodiments, the secondary shape may include the proximal section having a different shape than the distal section, with the shape of the proximal section being configured to collapse and pack onto itself in the vessel, thereby creating an improved pack to occlude the vessel. The proximal end may have a wave shape in some embodiments. In other embodiments, the proximal end or the entire embolic coil may be substantially oval, square, and/or have alternating diameter changes configured to create a tightly packed coil with minimal open space between secondary shape windings.

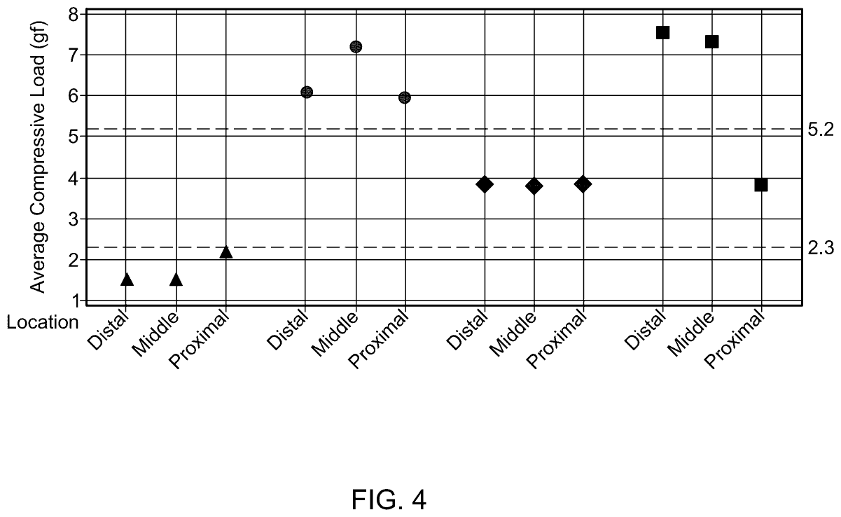

Coil compression force testing may be used to evaluate the amount of radial force that an embolization coil can produce when the embolization coil is being deployed in a blood vessel. Radial force or compressive load is a measure of an embolization coil's ability to anchor. FIG. 4 is a graph showing average compressive load, measured in gram force (gf) of the distal, middle, and proximal sections of various embolic coils. Results of the coil compression force testing are provided below in Table 1. Each coil had an outer diameter of 6 mm and length of 20 mm. The untreated coil (None) was made from a 0.003 inch (0.076 mm) diameter wire and the coils treated with IR radiation were made from a 0.004 inch (0.102 mm) diameter wire. The measurements of radial force are in grams-force (gf).

TABLE 1

| Heat Cycle | Distal | Middle | Proximal |
|---|---|---|---|
| None | 1.5 gf | 1.5 gf | 2.2 gf |
| IR full coil 1 minute | 6.1 gf | 7.2 gf | 6.0 gf |
| IR full coil 2 minutes | 3.9 gf | 3.8 gf | 3.9 gf |
| IR proximal coil | 7.6 gf | 7.3 gf | 4.0 gf |

In FIG. 4, the triangles on the left represent an EMBOLD™ Fibered Coil (Boston Scientific) with a size of 6 mm diameter and 20 cm length made from 0.003-inch diameter wire. The coil was annealed by exposing the entire coil to generally consistent IR radiation along the length of the coil. As shown in the graph, the entire coil has a similar stiffness, with an average radial force of 0.023 Newtons (N) (2.3 grams-force (gf)). To further show how embolic coil radial force can be increased, multiple 6 mm diameter and 20 cm long embolization coils were made with 0.102 mm (0.004-inch) diameter wire. Using the same manufacturing method as the EMBOLD™ Fibered Coil, with IR radiation over the entire coil, a mean radial force of 0.051 N (5.2 gf) was achieved, as shown by the circles and diamonds in the middle two groups. The circles represent a coil exposed to IR radiation over the entire coil for one minute, and the diamonds represent a coil exposed to IR radiation over the entire coil for two minutes. By varying the time parameter, the embolization coils were formed having greater radial force or less radial force, with longer times resulting in lower average compressive load (softer coil). The squares at the right side of the graph represent varying the parameter of the location of heat concentration. Concentrating the IR radiation source (lamp) on the proximal section of the coil results in the distal and the middle sections having a higher radial force (stiffer coil) compared to the proximal end of the coil.

The method of manufacturing the embolic coil may include exposing the secondary embolic coil on the mandrel to IR radiation focused on the proximal section of the coil, resulting in the distal and middle sections of the embolic coil exhibiting a radial force of approximately 0.074 N (7.5 gf) while the proximal section of the embolic coil exhibits a radial force of approximately 0.039 N (4.0 gf). In some embodiments, the embolic coil may be treated with IR radiation using a combination of time and location parameter variation to achieve a proximal section having a radial force of between 0.020 and 0.049 N (2 and 5 gf) while the distal and middle sections have a radial force of between 0.039 and 0.098 N (4 and 10 gf), always maintaining the distal section with a higher radial force than the proximal section. The focusing of the IR radiation may be achieved using a shield over the distal section as described above. The resulting embolic coil has a proximal section that is significantly softer with a lower average compressive load and radial force as compared to the middle and distal sections.

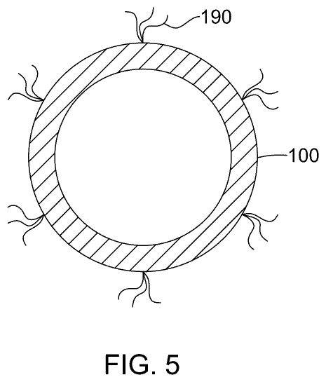

FIG. 5 is a cross-sectional view of another example embolic coil with a plurality of fibers attached. In some embodiments, the embolic coil 100 may include a plurality of fibers 190 attached to the windings of the coil, as shown in FIG. 5. The plurality of fibers 190 may be attached to the embolic coil 100 after IR treatment. The plurality of fibers 190 may be attached between windings of the embolic coil 100 by winding the middle part of each elongate fiber around the wire such that the middle section of each fiber 190 is sandwiched between adjacent windings with both free ends of the fiber 190 extending freely, as shown in FIG. 5. A bundle of multiple fibers 190 may be attached at each location. Each of the plurality of fibers 190 may be between 3 mm (0.12 inch) and 20 mm (0.78 inch) in total length, such that when attached to the embolic coil 100, the two free ends extend about 1.5 mm (0.06 inch) to 10 mm (0.4 inch) from the coil. The plurality of fibers 190 may be attached in a pattern or at random positions along the length of the embolic coil 100, and multiple fibers 190 may be attached at a single location, creating a bundle of fibers. In some embodiments, the plurality of fibers 190 may be attached only to a proximal section of the embolic coil 100. In other embodiments, the plurality of fibers 190 may be attached along the entirety of the length of the embolic coil 100. The plurality of fibers 190 may be attached at random locations around the entire circumference of the embolic coil 100. The plurality of fibers 190 may be made of thrombogenic fibers which enhance thrombosis. In some embodiments, the plurality of fibers 190 may be formed from a polymer, including at least one of polyester, nylon, and polypropylene. In some embodiments, a plurality of fibers 190 may be attached to the embolic coil 100, after IR radiation treatment in the secondary shape. The embolic coil 100 may be devoid of any structure on its outermost surface other than the plurality of fibers 190.

A method of using the embolic coil 50,100 described above may include loading the embolic coil 50, 100 into a delivery catheter and inserting the delivery catheter into a patient's vasculature to a treatment site. At the treatment site, the embolic coil 50, 100 may be pushed out of the delivery catheter using a delivery wire which may then be uncoupled from the embolic coil 50, 100, or the delivery catheter may be withdrawn proximally leaving the embolic coil 50, 100 in place at the treatment site, where the embolic coil 50, 100 moves into the secondary shape. In some embodiments, a flowable material may be delivered with a single embolic coil 50, 100 such as, but not limited to OBSIDIO™ Conformable Embolic, available from Boston Scientific of Marlborough, MA. USA. The flowable material may be delivered after the embolic coil 50, 100 has been released from the delivery catheter and assumes its secondary shape, if it has one. Some embolic coils 50, 100 may not have a secondary shape, and may be treated with IR radiation in their primary shape.

It will be understood that the dimensions described in association with the above figure are illustrative only, and that other dimensions are contemplated. The materials that can be used for the various components of the embolic coil 50, 100 and delivery system and the various elements thereof disclosed herein may include those commonly associated with medical devices. For simplicity purposes, the following discussion refers to the embolic coil 50, 100 (and variations, systems or components disclosed herein). However, this is not intended to limit the devices and methods described herein, as the discussion may be applied to other elements, members, components, or devices disclosed herein.

In some embodiments, the embolic coil 50, 100 or fibers 190 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An embolic coil comprising:
a single wire formed into a primary coil having a primary shape and a constant diameter along an entirety of its length, the single wire having a constant diameter along an entirety of its length, the primary coil formed into a secondary coil having a secondary shape different from the primary shape with a distal section and a proximal section, the distal section of the secondary coil having a first stiffness and the proximal section having a second stiffness less than the first stiffness; and
wherein a difference in stiffness between the distal and proximal sections is created by selective infrared heat treatment.

2. The embolic coil of claim 1, wherein the single wire comprises a platinum-tungsten alloy.

3. The embolic coil of claim 1, wherein the first stiffness is approximately double the second stiffness.

4. The embolic coil of claim 1, wherein the secondary coil is treated with infrared radiation in the secondary shape to create the difference in stiffness between the distal and proximal sections.

5. The embolic coil of claim 1, further comprising a plurality of polymeric fibers attached to the secondary coil.

6. A method of manufacturing an embolic coil comprising:
forming a single wire into a primary coil;
forming the primary coil into a secondary coil having a distal section and a proximal section; and
selectively applying infrared radiation to the secondary coil to create a first stiffness in the distal section and a second stiffness in the proximal section, wherein the first stiffness is greater than the second stiffness.

7. The method of claim 6, wherein selectively applying infrared radiation comprises:
exposing the distal section to a first amount of infrared radiation; and
exposing the proximal section to a second amount of infrared radiation greater than the first amount of infrared radiation.

8. The method of claim 6, wherein selectively applying infrared radiation comprises exposing the proximal section to infrared radiation for a first time period that is approximately twice as long as a second time period in which the distal section is exposed to infrared radiation.

9. The method of claim 6, wherein selectively applying infrared radiation includes positioning a reflective shield over the distal section of the secondary coil for at least a first time period while exposing the secondary coil to infrared radiation such that the reflective shield prevents absorption of the infrared radiation by the distal section of the secondary coil during infrared radiation exposure.

10. The method of claim 6, wherein the single wire is made of a platinum-tungsten alloy.

11. The method of claim 6, wherein the primary coil has a constant diameter along an entirety of its length.

12. The method of claim 6, wherein the single wire has a diameter of 0.076 mm (0.003 inches) or 0.102 mm (0.004 inches).

13. The method of claim 6, wherein the proximal section exhibits a first radial force of approximately 0.039 N (4 gf) and the distal section exhibits a second radial force of approximately 0.074 N (7.5 gf).

14. The method of claim 6, wherein the single wire has a constant diameter along an entirety of its length.

15. The method of claim 6, wherein the first stiffness is approximately double the second stiffness.

16. The method of claim 6, wherein selectively applying infrared radiation occurs when the secondary coil is disposed on a mandrel.

17. The method of claim 16, wherein the mandrel has a hollow core.

18. The method of claim 6, further comprising attaching a plurality of polymeric fibers to the secondary coil after selectively applying infrared radiation to the secondary coil.

19. A method of manufacturing an embolic coil comprising:
forming a single wire into a primary coil, the single wire having a constant diameter along an entirety of its length, the primary coil having a constant outer diameter along an entirety of its length;
forming the primary coil into a secondary coil having a distal section and a proximal section; and
selectively applying infrared radiation to the secondary coil to create a first stiffness in the distal section and a second stiffness in the proximal section, wherein the first stiffness is greater than the second stiffness;
wherein selectively applying infrared radiation includes exposing the proximal section of the secondary coil to infrared radiation for a longer time period than the distal section.

20. The method of claim 19, wherein exposing the proximal section of the secondary coil to infrared radiation for the longer time period is achieved by positioning a shield member over the distal section of the secondary coil during at least a portion of the longer time period.
